(19) United States
(12) Patent Application Publication
(10) Pub. No.: US 2024/0368076 A1
(43) Pub. Date: Nov. 7, 2024

(54) COMPOUND, CURABLE RESIN COMPOSITION, CURED PRODUCT, DIFFRACTIVE OPTICAL ELEMENT, AND AUGMENTED REALITY GLASSES

(71) Applicant: FUJIFILM Corporation, Tokyo (JP)

(72) Inventors: Naozumi SHIRAIWA, Ashigarakami-gun (JP); Naoyuki MOROOKA, Ashigarakami-gun (JP)

(73) Assignee: FUJIFILM Corporation, Tokyo (JP)

(21) Appl. No.: 18/740,684

(22) Filed: Jun. 12, 2024

Related U.S. Application Data
(63) Continuation of application No. PCT/JP2023/004956, filed on Feb. 14, 2023.

(30) Foreign Application Priority Data
Feb. 25, 2022 (JP) 2022-028508

(51) Int. Cl. C07C 327/22; C08F 120/68; G02B 5/18

(52) U.S. Cl. CPC C07C 327/22; C08F 120/68; G02B 5/18

(57) ABSTRACT

Provided are a compound represented by General Formula (1), a curable resin composition containing the compound, a cured product formed of the curable resin composition, a diffractive optical element, and augmented reality glasses.

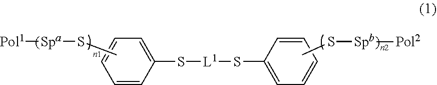

In the formula, ^{L} represents an alkylene group or a group that, among one —CH₂— or two or more —CH₂—'s not adjacent to each other in an alkylene group, —CH₂— not adjacent to an S atom is substituted with —S—,
Sp^{a} and Sp^{b} represent a divalent aromatic hydrocarbon group or a divalent group formed by a combination of a divalent aromatic hydrocarbon group and an alkylene group,
Pol¹ and Pol² represent a (meth)acryloylthio group or a vinylthio group, and
n1 and n2 are 0 or 1.

### CROSS-REFERENCE TO RELATED APPLICATIONS

This application is a Continuation of PCT International Application No. PCT/JP2023/004956 filed on Feb. 14, 2023, which claims priority under 35 U.S.C. § 119 (a) to Japanese Patent Application No. 2022-028508 filed in Japan on Feb. 25, 2022. Each of the above applications is hereby expressly incorporated by reference, in its entirety, into the present application.

### BACKGROUND OF THE INVENTION

### 1. Field of the Invention

The present invention relates to a compound, a curable resin composition, a cured product, a diffractive optical element, and augmented reality glasses.

### 2. Description of the Related Art

As one of display methods of an augmented reality (AR) glass, which is expected as a next-generation, a waveguide method of changing an angle of ray within a lens itself to project an image has been known.

In a diffractive optical element (DOE) used in the waveguide method, a diffraction grating is formed by a nanofabrication technology in which a mold having a pattern in a nanometer unit, which is called nanoimprint, is embossed on a material such as a resin, and the pattern in a nanometer unit is transferred to a surface of the material such as a resin.

In order to restore the fine pattern in a nanometer unit with high accuracy, it is required that the material such as a resin, which is used for the nanoimprinting, does not contain inorganic particles and has low viscosity.

From the viewpoint of expanding a viewing angle of the AR display, the resin constituting the DOE for a waveguide method is required to have a high refractive index.

For example, JP1990-193962A (JP-H2-193962A) discloses a production method of 4,4′-bis(4-methacryloylthiophenyl)sulfide which is useful as a raw material of a high refractive index transparent resin.

In addition, JP1993-51412A (JP-H5-51412A) discloses an organic glass exhibiting a high refractive index, which is obtained by a bulk polymerization of a monomer having a disulfide bond and a radically polymerizable unsaturated bond in one molecule.

### SUMMARY OF THE INVENTION

The nanoimprint includes a step of applying a material onto a substrate, a step of pressing a mold in a state of being embossed, a step of transferring by photocuring or thermal curing, and a step of demolding. The material used in the nanoimprint is temporarily stored in a tank and used as in a case of an ink in an ink jet, until the above-described nanoimprint process is performed, so that it is required that crystals are not precipitated in the storage tank. In addition, a cured product obtained by the photocuring or thermal curing is required to have properties that can withstand deterioration over time, for example, have heat resistance.

As a result of studies by the present inventors, with regard to the polymerizable compound disclosed in JP1990-193962A (JP-H2-193962A), in a case where a content of the polymerizable compound in a curable resin composition containing a polymerizable compound, a polymerization initiator, a monomer for dilution, and the like is increased to obtain a cured product having a high refractive index required for the DOE for a waveguide method, precipitation of crystals is likely to occur, and it has been found that it is impossible to achieve both the high refractive index of the cured product and the suppression of the crystal precipitation of the composition.

In addition, it has been found that, since the compound disclosed in JP1993-51412A (JP-H5-51412A) has the disulfide bond, a cured product to be obtained has low heat resistance and is likely to be cracked.

An object of the present invention is to provide a compound with which a cured product having a high refractive index and excellent heat resistance can be obtained and in which crystal precipitation in a case of being used in a curable resin composition is suppressed. Another object of the present invention is to provide a curable resin composition containing the compound, a cured product obtained from this curable resin composition, and a diffractive optical element and augmented reality glasses, including this cured product.

As a result of intensive studies to achieve the above-described objects, the present inventors have found that, by using a polymerizable compound in which two phenylenethio groups are linked by a specific alkylene group, it is possible to suppress the crystal precipitation in a case of being used in a curable resin composition, while achieving a high refractive index and excellent heat resistance of the cured product to be obtained.

That is, the above-described objects of the present invention have been achieved by the following methods.

<1>

A compound represented by General Formula (1),

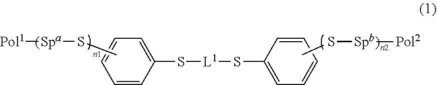

in the formula, L¹ represents an alkylene group or a group that, among one —CH₂— or two or more —CH₂—'s not adjacent to each other in an alkylene group, —CH₂— not adjacent to an S atom is substituted with —S—,
Sp^{a} and Sp^{b} represent a divalent aromatic hydrocarbon group or a divalent group formed by a combination of a divalent aromatic hydrocarbon group and an alkylene group,
Pol¹ and Pol² represent a (meth)acryloylthio group or a vinylthio group, and
n1 and n2 are 0 or 1.
   <2>

The compound according to <1>,
in which L¹ has a number of linking atoms of 1 to 5, the linking atoms constituting a shortest molecular chain linking two sulfur atoms to which both terminals of L¹ are bonded.
   <3>

The compound according to <1> or <2>,
in which n1 and n2 are 1.
   <4>

A curable resin composition comprising:
the compound according to any one of <1> to <3>.
   <5>

The curable resin composition according to <4>,
in which a content of the compound according to any one of <1> to <3> in the curable resin composition is 60% by mass or more.
   <6>

The curable resin composition according to <4> or <5>,
in which the curable resin composition is for nanoimprint.
   <7>

A cured product of the curable resin composition according to any one of <4> to <6>.

<8>

A diffractive optical element which is formed of the cured product according to <7>, comprising:
a surface having a diffraction grating shape.
   <9>

An augmented reality glasses comprising:
the diffractive optical element according to <8>.

In the present invention, in a case of a plurality of substituents, linking groups, or the like (hereinafter, referred to as a substituent or the like) represented by a specific reference numeral or formula, or in a case of simultaneously defining a plurality of the substituent and the like, unless otherwise specified, the substituent and the like may be the same or different from each other (regardless of the presence or absence of an expression “each independently”, the substituent and the like may be the same or different from each other). The same applies to the definition of the number of substituents and the like. In a case where a plurality of substituents and the like are near (particularly, adjacent to each other), unless otherwise specified, the substituents and the like may be linked to each other to form a ring. In addition, unless otherwise specified, a ring, for example, an alicyclic ring, an aromatic ring, or a heterocyclic ring may be further condensed to form a fused ring.

In the present invention, unless otherwise specified, with regard to a double bond, in a case where E-form and Z-form are present in the molecule, the double bond may be any one of these forms, or may be a mixture thereof.

In addition, in the present invention, unless otherwise specified, in a case where a compound has one or two or more asymmetric carbons, for such stereochemistry of asymmetric carbons, either an (R)-form or an (S)-form can be independently taken. As a result, the compound may be a mixture of optical isomers or stereoisomers such as diastereoisomers, or may be racemic.

In addition, in the present invention, the expression of the compound means that a compound having a partially changed structure is included within a range which does not impair the effects of the present invention. Furthermore, a compound which is not specifically described as substituted or unsubstituted may have an optional substituent within a range which does not impair the effects of the present invention. For example, in the compound represented by General Formula (1), L¹, Sp^{a}, Sp^{b}, and a benzene ring may be unsubstituted or may have an optional substituent.

In the present invention, with regard to a substituent (the same applies to a linking group and a ring) in which whether it is substituted or unsubstituted is not specified, within a range not impairing the desired effect, it means that the group may have an optional substituent. For example, “alkyl group” means to include both an unsubstituted alkyl group and a substituted alkyl group. The same applies to “alkylene group” and “divalent aromatic hydrocarbon group”.

In the present invention, in a case where the number of carbon atoms in a certain group is specified, the number of carbon atoms means the number of carbon atoms in the entire group, unless otherwise specified in the present invention or the present specification. That is, in a case of a form in which the group has a substituent, it means the total number of carbon atoms including the substituent.

In the present invention, a numerical range represented by using “to” means a range including numerical values described before and after “to” as a lower limit value and an upper limit value.

In the curable resin composition according to the aspect of the present invention, each component may be used alone or in combination of two or more thereof. The same applies to the cured product, the diffractive optical element, and the augmented reality glasses obtained from the curable resin composition according to the embodiment of the present invention.

In the present invention, “(meth)acrylate” represents either one or both of acrylate and methacrylate, and “(meth)acryloyl” represents either one or both of acryloyl and methacryloyl. The monomer in the present invention is distinguished from an oligomer and a polymer, and refers to a compound having a weight-average molecular weight of 1000 or less.

In the present invention, the term alkyl group represents a linear or branched alkyl group.

The number of carbon atoms in the alkyl group is preferably 1 to 20, more preferably 1 to 10, still more preferably 1 to 7, and particularly preferably 1 to 5.

Examples of the alkyl group include a methyl group, an ethyl group, a propyl group, an isopropyl group, a butyl group, an isobutyl group, a sec-butyl group, a tert-butyl group, a pentyl group, a 1-methylbutyl group, a 3-methylbutyl group, a hexyl group, a 1-methylpentyl group, a 4-methylpentyl group, a heptyl group, a 1-methylhexyl group, a 5-methylhexyl group, a 2-ethylhexyl group, an octyl group, a 1-methylheptyl group, a nonyl group, a 1-methyloctyl group, a decyl group, an undecyl group, a dodecyl group, a tridecyl group, a tetradecyl group, a pentadecyl group, a hexadecyl group, a heptadecyl group, an octadecyl group, a nonadecyl group, and an eicosyl group; and among these, a methyl group or an ethyl group is preferable.

The same applies to an alkyl group in a group (an alkoxy group, an alkoxycarbonyl group, an acyl group, an acyloxy group, and the like) including the alkyl group.

In addition, the alkyl group may have a substituent, and examples of such an alkyl group having a substituent include a halogenated alkyl group and a hydroxyalkyl group.

In the present invention, as an alkenyl group, an alkenyl group having 2 to 6 carbon atoms is preferable, and examples thereof include a vinyl group and an allyl group.

In the present invention, examples of an alkylene group include a group obtained by removing one hydrogen atom bonded to a carbon atom in the above-described alkyl group, and the alkylene group may be a linear alkylene group or a branched alkylene group. Examples thereof include an ethylene group, a propylene group, and a butylene group.

In the present invention, a cycloalkyl group refers to a monovalent group obtained by removing one optional hydrogen atom from a cycloalkane. The cycloalkyl group is preferably a cycloalkyl group having 3 to 10 carbon atoms, and examples thereof include a cyclobutyl group, a cyclopentyl group, and a cyclohexyl group.

In the present invention, a monovalent aromatic hydrocarbon group represents a monovalent group obtained by removing any one hydrogen atom from an aromatic hydrocarbon ring which may be a single ring or a fused ring. As the monovalent aromatic hydrocarbon group is preferably an aromatic hydrocarbon group having 6 to 14 carbon atoms, and examples thereof include a phenyl group, an 1-naphthyl groups, a 2-naphthyl groups, an 1-anthracenyl group, a 2-anthracenyl group, a 9-anthracenyl group, an 1-phenanthryl group, a 2-phenanthryl group, a 3-phenanthryl group, a 4-phenanthryl group, and a 9-phenanthryl group. Among these, a phenyl group is preferable.

In the present invention, a divalent aromatic hydrocarbon group represents a divalent group obtained by removing any one hydrogen atom from the above-described monovalent aromatic hydrocarbon group. Examples of the divalent aromatic hydrocarbon group include a phenylene group, a naphthylene group, and a phenanthrylene group, and a phenylene group is preferable and a 1,3-phenylene group or a 1,4-phenylene group is more preferable.

In the present invention, an aromatic heterocyclic ring means an aromatic ring in which a ring is formed by a carbon atom and a heteroatom. Examples of the heteroatom include an oxygen atom, a nitrogen atom, and a sulfur atom. The aromatic heterocyclic ring may be a monocyclic ring or a fused ring, and the number of atoms constituting the ring is preferably 5 to 20 and more preferably 5 to 14. The number of heteroatoms in the atoms constituting the ring is not particularly limited, but is preferably 1 to 3 and more preferably 1 or 2. Examples of the aromatic heterocyclic ring include a furan ring, a thiophene ring, a pyrrole ring, an imidazole ring, an isothiazole ring, an isoxazole ring, a pyridine ring, a pyrazine ring, a quinoline ring, a benzofuran ring, a benzothiazole ring, a benzoxazole ring, and examples of nitrogen-containing fused aromatic ring, which will be described later. In the present invention, in a case where the aromatic heterocyclic ring is bonded to another ring, it is sufficient that the aromatic heterocyclic ring may be substituted on another ring as a monovalent or divalent aromatic heterocyclic group.

In the present invention, a monovalent aromatic heterocyclic group represents a monovalent group obtained by removing any one hydrogen atom from an aromatic heterocyclic ring. Examples of the monovalent aromatic heterocyclic group include a furyl group, a thienyl group, a pyrrolyl group, an imidazolyl group, an isothiazolyl group, an isooxazolyl group, a pyridyl group, a pyrazinyl group, a quinolyl group, a benzofuranyl group (preferably, a 2-benzofuranyl group), a benzothiazolyl group (preferably, a 2-benzothiazolyl group), and a benzoxazolyl group (preferably, a 2-benzoxazolyl group). Among these, a furyl group, a thienyl group, a benzofuranyl group, a benzothiazolyl group, or a benzoxazolyl group is preferable, and a 2-furyl group or a 2-thienyl group is more preferable.

In the present invention, the divalent aromatic heterocyclic group refers to a divalent group obtained by removing two optional hydrogen atoms from the aromatic heterocyclic ring, and examples thereof include a divalent group obtained by removing one optional hydrogen atom from the above-described (monovalent) aromatic heterocyclic group.

In the present invention, examples of a halogen atom include a fluorine atom, a chlorine atom, a bromine atom, and an iodine atom.

With the compound according to the aspect of the present invention, crystal precipitation in a case of being used in a curable resin composition is suppressed, and a cured product having a high refractive index and excellent heat resistance can be obtained. In addition, with the curable resin composition according to the aspect of the present invention, crystal precipitation is suppressed, it is possible to achieve a high refractive index and excellent heat resistance of a cured product obtained by curing the curable resin composition. The cured product and the diffractive optical element according to the aspect of the present invention can exhibit a high refractive index and excellent heat resistance. The augmented reality glasses according to the embodiment of the present invention include a diffractive optical element which has a high refractive index and excellent heat resistance.

### DESCRIPTION OF THE PREFERRED EMBODIMENTS

### [Compound]

The compound according to the embodiment of the present invention is represented by General Formula (1).

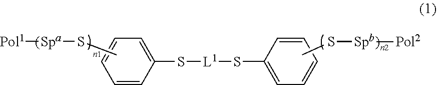

In the formula, L¹ represents an alkylene group or a group that, among one —CH₂— or two or more —CH₂—'s not adjacent to each other in an alkylene group, —CH₂— not adjacent to an S atom is substituted with —S—,
Sp^{a} and Sp^{b} represent a divalent aromatic hydrocarbon group or a divalent group formed by a combination of a divalent aromatic hydrocarbon group and an alkylene group,
Pol¹ and Pol² represent a (meth)acryloylthio group or a vinylthio group, and
n1 and n2 are 0 or 1.

As shown in General Formula (1), the compound according to the embodiment of the present invention is a compound in which a benzene ring having a polymerizable group Pol¹ as a substituent and a benzene ring having a polymerizable group Pol² as a substituent are linked to each other through a linking group represented by —S-L¹-S—.

Since the compound according to the embodiment of the present invention has a sulfur atom, a cured product to be obtained can exhibit a high refractive index. Furthermore, it is presumed that, with a chemical structure in which an alkylene group is adopted as L¹, or with a chemical structure in which a group that, among one —CH₂— or two or more —CH₂—'s not adjacent to each other in an alkylene group, —CH₂— not adjacent to an S atom which is bonded to L¹ in General Formula (1) is substituted with —S— is adopted as L¹, molecular mobility is improved and crystal precipitation of a curable resin composition containing the compound according to the embodiment of the present invention is suppressed. In addition, since L¹ does not have a structure in which two or more S's are linked, it is considered that the compound can exhibit excellent heat resistance as compared with the compound in the related art, such as the compound disclosed in JP1993-51412A (JP-H5-51412A), in which two groups having a polymerizable group are linked to each other through a linking group containing a disulfide bond.

In this manner, with the compound according to the embodiment of the present invention, crystal precipitation in a case of being used in a curable resin composition is suppressed, and a cured product having a high refractive index and excellent heat resistance can be obtained.

### (1) L¹

L¹ represents an alkylene group or a group that, among one —CH₂— or two or more —CH₂— not adjacent to each other in an alkylene group, —CH₂— not adjacent to an S atom is substituted with —S—.

In the present invention, the “L¹ represents a group that, among one —CH₂— or two or more —CH₂— not adjacent to each other in an alkylene group, —CH₂— not adjacent to an S atom is substituted with —S—” means that L¹ is a group which has a —S— bond in an alkylene group and does not have, as the group represented by —S-L¹-S— in General Formula (1), a structure in which two or more S's are linked (a disulfide structure or a polysulfide structure in which three or more S's are linked).

The number of carbon atoms in the alkylene group which can be adopted as L¹ is preferably 1 to 10, more preferably 1 to 7, and still more preferably 1 to 5. The alkylene group may be linear or branched. The alkylene group may or may not have a cyclic structure, and it is preferable that the alkylene group does not have a cyclic structure.

In the group that, among one —CH₂— or two or more —CH₂— not adjacent to each other in an alkylene group, —CH₂— not adjacent to an S atom is substituted with —S—, which can be adopted as L¹, with regard to the alkylene group before being substituted with —S—, the above description of the alkylene group which can be adopted as L¹ can be applied.

In the group that, among one —CH₂— or two or more —CH₂— not adjacent to each other in an alkylene group, —CH₂— not adjacent to an S atom is substituted with —S—, which can be adopted as L¹, the number of —CH₂—'s substituted with —S— is not particularly limited.

L¹ is preferably alkylene, -alkylene-S-alkylene-, or -alkylene-S-alkylene-S-alkylene-, and more preferably alkylene or -alkylene-S-alkylene-. The number of carbon atoms in an alkylene portion of the -alkylene-S-alkylene- or the -alkylene-S-alkylene-S-alkylene- is preferably 1 to 3 and more preferably 1 or 2.

From the viewpoint of further increasing a refractive index nD of the cured product, the number of linking atoms constituting the shortest molecular chain linking two sulfur atoms to which both terminals of L¹ are bonded is preferably 1 to 10, more preferably 1 to 7, and still more preferably 1 to 5.

As an example of the “number of linking atoms constituting the shortest molecular chain linking two sulfur atoms to which both terminals of L¹ are bonded”, in a compound A-10 shown below, the number of linking atoms constituting the shortest molecular chain linking two sulfur atoms to which both terminals of L¹ are bonded is 5.

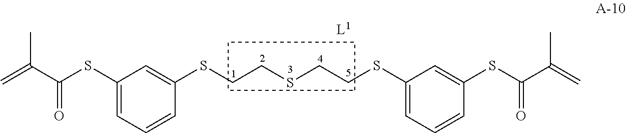 [SMILES: C=C(C)C(=O)Sc1cccc(SCCSCCSc2cccc(SC(=O)C(=C)C)c2)c1]

(2) Sp^{a} and Sp^{b}

Sp^{a} and Sp^{b} represent a divalent aromatic hydrocarbon group or a divalent group formed by a combination of a divalent aromatic hydrocarbon group and an alkylene group.

The number of carbon atoms in the divalent aromatic hydrocarbon group which can constitute Sp^{a} and Sp^{b} is preferably 6 to 10 and more preferably 6.

The number of carbon atoms in the alkylene group which can constitute Sp^{a} and Sp^{b} is preferably 1 to 6, more preferably 1 to 4, still more preferably 1 or 2, and particularly preferably 1.

In the divalent group formed by a combination of a divalent aromatic hydrocarbon group and an alkylene group, which can be adopted as Sp^{a} and Sp^{b}, the number of divalent aromatic hydrocarbon groups and the number of alkylene groups are not particularly limited, and for example, each can be 1 to 3. Among these, a divalent aromatic hydrocarbon-alkylene group is preferable.

In a case where Sp^{a} and Sp^{b} are the divalent aromatic hydrocarbon-alkylene group, Sp^{a} and Sp^{b} may be bonded to Pol¹ or Pol² on any one side of the divalent aromatic hydrocarbon group or the alkylene group, and it is preferable to be bonded to Pol¹ or Pol² on the alkylene group side.

Sp^{a} and Sp^{b} are preferably a divalent aromatic hydrocarbon group having 6 to 10 carbon atoms or a divalent group formed by a combination of a divalent aromatic hydrocarbon group having 6 to 10 carbon atoms and an alkylene group having 1 to 6 carbon atoms, more preferably a phenylene group, a phenylenemethylene group, or a phenyleneethylene group, and still more preferably a phenylene group or a phenylmethylene group.

(3) Pol¹ and Pol²

Pol¹ and Pol² represent a (meth)acryloylthio group or a vinylthio group, and are each a polymerizable group represented by Formulae (Pol-1) to (Pol-3).

Among these, an acryloylthio group represented by Formula (Pol-1) or a methacryloylthio group represented by Formula (Pol-2) is preferable.

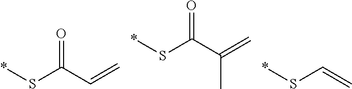

Formula (Pol-1) Formula (Pol-2) Formula (Pol-3) *represents a bonding position.
   (4) n1 and n2

n1 and n2 are 0 or 1 (that is, an integer of 0 or 1).

From the viewpoint of further increasing the refractive index nD of the cured product, it is preferable that n1 and n2 are 1.

Hereinafter, preferred specific examples of the compound represented by General Formula (1) are listed, but the present invention is not limited to these compounds. In the following chemical structures, * represents a bonding site.

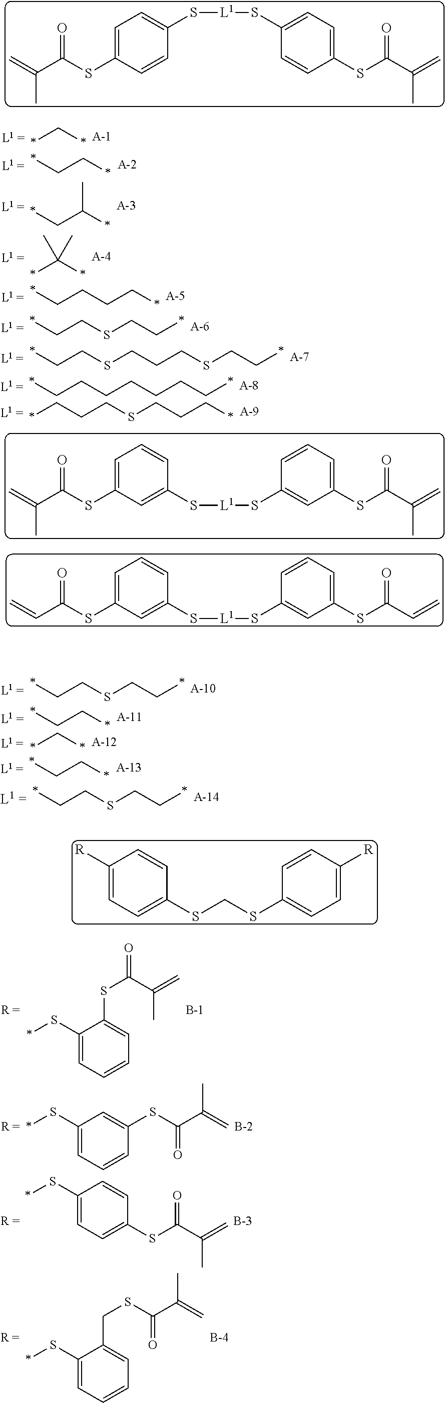

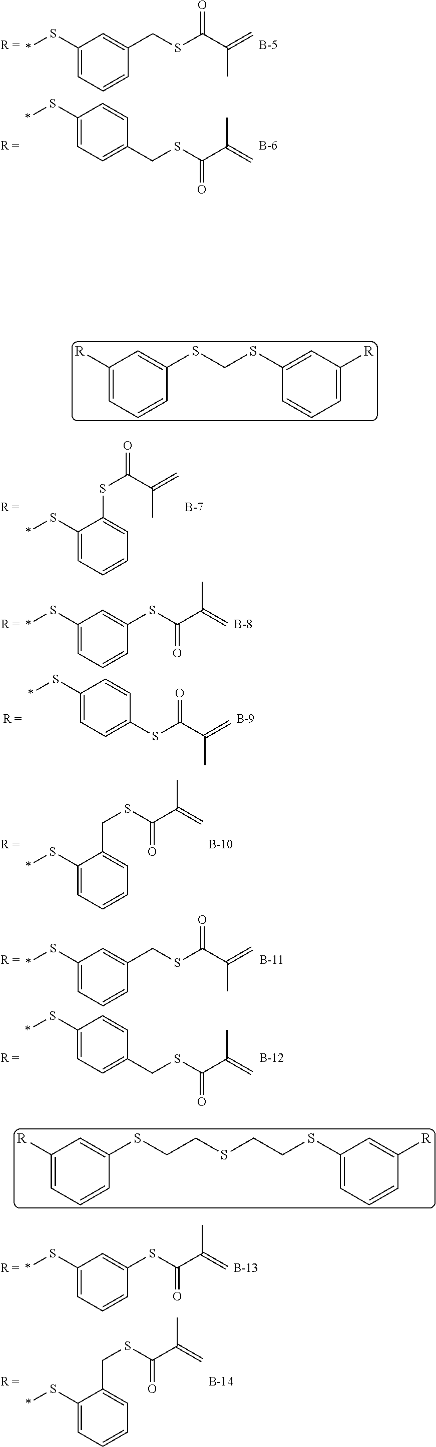

A molecular weight of the compound represented by General Formula (1) is preferably 348 to 900, more preferably 348 to 800, and particularly preferably 348 to 700.

The compound represented by General Formula (1) can be synthesized by a common method. For example, the compound can be synthesized with reference to a synthesis method described in Org. Lett., 2004, 6, 4587, a synthesis method described in US2003/0195270A, and the like. In addition, the compound can be synthesized as appropriate with reference to methods described in Examples, and the like.

### [Curable Resin Composition]

The curable resin composition according to the embodiment of the present invention contains the compound represented by General Formula (1).

The curable resin composition according to the embodiment of the present invention means a composition which has curing properties and with which a cured product (resin) can be obtained by a curing reaction.

In the curable resin composition according to the embodiment of the present invention, crystal precipitation is suppressed, and a cured product having a high refractive index and excellent heat resistance can be obtained by curing the curable resin composition according to the embodiment of the present invention.

A content of the compound represented by General Formula (1) in the curable resin composition according to the embodiment of the present invention can be, for example, 60% by mass or more, and from the viewpoint of further improving nD (a refractive index at a wavelength of 589 nm), it is preferably 70% by mass or more and more preferably 80% by mass or more. An upper limit value of the above-described content of the compound represented by General Formula (1) is not particularly limited, and the content of the compound represented by General Formula (1) in a curable resin composition containing a polymerization initiator described later and the compound represented by General Formula (1) can be, for example, 99.99% by mass or less, preferably 99.95% by mass or less, more preferably 99.90% by mass or less, and still more preferably 99.7% by mass or less. The content of the compound represented by General Formula (1) in the curable resin composition according to the embodiment of the present invention can be, for example, 60% to 99.99% by mass, preferably 70% to 99.95% by mass, more preferably 80% to 99.90% by mass, and still more preferably 80% to 99.7% by mass.

In a case where the content of the compound represented by General Formula (1) in the curable resin composition according to the embodiment of the present invention is increased, for example, in a case of 99.7% by mass, the crystal precipitation can be sufficiently suppressed, which is preferable.

The above-described curable resin composition may contain one kind of the compound represented by General Formula (1), or two or more kinds thereof. In a case of containing two or more kinds of the compounds represented by General Formula (1), the total content thereof is preferably within the above-described range.

### <Other Components>

The curable resin composition according to the embodiment of the present invention is a mixture containing, in addition to the compound represented by General Formula (1), other components. Examples of the other components include a (meth)acrylate monomer, a polymer having a radically polymerizable group in the side chain, and a polymerization initiator.

### ((Meth)Acrylate Monomer)

The curable resin composition according to the embodiment of the present invention may contain a (meth)acrylate monomer other than the above-described compound represented by General Formula (1), as a monomer for dilution. The (meth)acrylate monomer may be a polyfunctional (meth)acrylate monomer having two or more (meth)acryloyl groups in the molecule, or may be a monofunctional (meth)acrylate monomer having one (meth)acryloyl group in the molecule. The (meth)acryloyl group is preferably included in the (meth)acrylate monomer in a form of a (meth)acryloyloxy group or a (meth)acryloylthio group.

Specific examples of the (meth)acrylate monomer include M-1 (benzyl acrylate), M-2 (benzyl methacrylate), M-3 (isobornyl methacrylate), M-4 (dicyclopentanyl acrylate), M-5 (dicyclopentanyl methacrylate), M-6 (phenoxyethyl acrylate), M-7 (2-ethylhexyl acrylate), M-8 (1,6-hexanediol diacrylate), M-9 (bis(2-acryloylthioethyl)sulfide), and M-10 (bis(2-methacryloylthioethyl)sulfide), which are shown below. In addition, examples thereof include (meth)acrylate monomers described in paragraphs 0037 to 0046 of JP2012-107191A.

A molecular weight of the (meth)acrylate monomer is preferably 100 to 500.

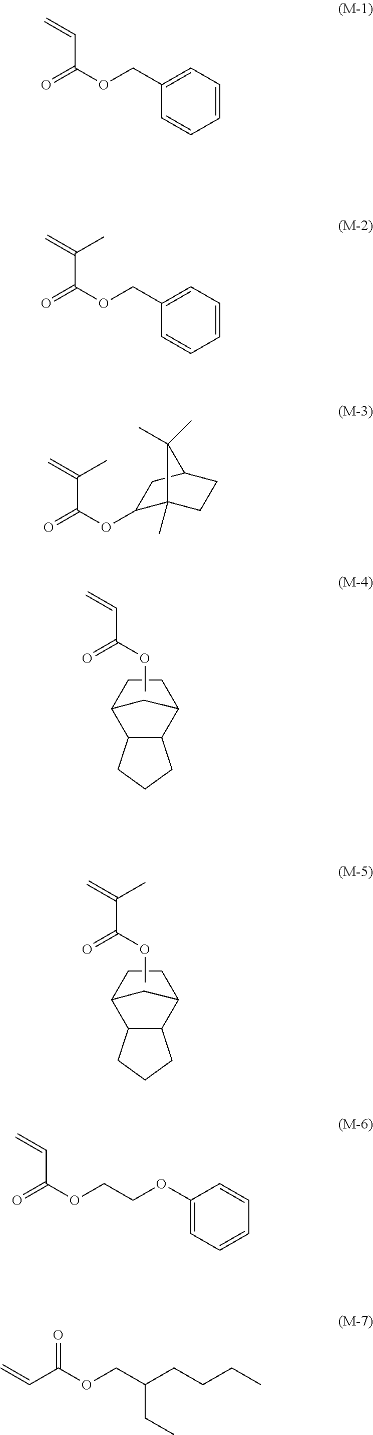

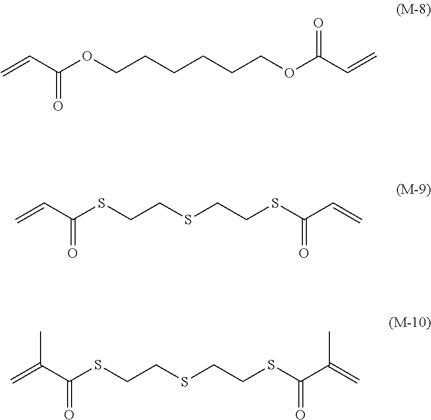

A method for obtaining the above-described (meth)acrylate monomer is not particularly limited, and the (meth)acrylate compound may be obtained commercially or may be synthesized by a conventional method.

In a case where the curable resin composition according to the embodiment of the present invention contains the above-described (meth)acrylate monomer, a content of the (meth)acrylate monomer in the curable resin composition is preferably 40% by mass or less, more preferably 35% by mass or less, still more preferably 30% by mass or less, and particularly preferably 25% by mass or less. By adjusting the amount of the (meth)acrylate monomer in the curable resin composition, it is possible to adjust a function of relieving a stress in a case where the cured product undergoes a thermal change. A lower limit value of the content of the (meth)acrylate monomer is not particularly limited, but for example, it is preferably 1% by mass or more, more preferably 5% by mass or more, still more preferably 10% by mass or more, and particularly preferably 15% by mass or more. The content of the (meth)acrylate monomer in the curable resin composition according to the embodiment of the present invention is, for example, preferably 1% to 40% by mass, more preferably 5% to 35% by mass, still more preferably 10% to 30% by mass, and particularly preferably 15% to 25% by mass.

### (Polymerization Initiator)

The curable resin composition according to the embodiment of the present invention preferably contains, as the polymerization initiator, at least one of a thermal radical polymerization initiator or a photoradical polymerization initiator. With the curable resin composition according to the embodiment of the present invention, a cured product having a high refractive index and excellent heat resistance can be obtained by a photopolymerization by an action of a photoradical polymerization initiator or a thermal polymerization by an action of a thermal radical polymerization initiator.

### (Thermal Radical Polymerization Initiator)

The curable resin composition according to the embodiment of the present invention preferably includes a thermal radical polymerization initiator. As the thermal radical polymerization initiator, a compound usually used as a thermal radical polymerization initiator can be appropriately used according to conditions of a thermopolymerization (heat curing) step described later. Examples thereof include organic peroxides, and specifically, the following compounds can be used.

Examples thereof include 1,1-di(t-hexylperoxy) cyclohexane, 1,1-di(t-butylperoxy) cyclohexane, 2,2-di(4,4-di-(t-butylperoxy)cyclohexyl) propane, t-hexylperoxyisopropyl monocarbonate, t-butylperoxy-3,5,5-trimethylhexanoate, t-butylperoxylaurate, dicumyl peroxide, di-t-butyl peroxide, t-butylperoxy-2-ethylhexanoate, di-t-hexylperoxide, t-hexylperoxy-2-ethylhexanoate, cumene hydroperoxide, t-butyl hydroperoxide, t-butylperoxy-2-ethylhexyl, and 2,3-dimethyl-2,3-diphenylbutane.

In a case of containing a thermal radical polymerization initiator, a content of the thermal radical polymerization initiator in the curable resin composition according to the embodiment of the present invention is preferably 0.01% to 5.0% by mass, more preferably 0.05% to 5.0% by mass, and still more preferably 0.05% to 2.0% by mass.

### (Photoradical Polymerization Initiator)

The curable resin composition according to the embodiment of the present invention preferably contains a photoradical polymerization initiator. As the photoradical polymerization initiator, a compound usually used as a photoradical polymerization initiator can be appropriately used according to conditions of a photopolymerization (photocuring) step described later, and specifically, the following compounds can be used.

Examples thereof include bis(2,6-dimethoxybenzoyl)-2,4,4-trimethylpentyl phosphine oxide, bis(2,6-dimethylbenzoyl)-2,4,4-trimethylpentyl phosphine oxide, bis(2,4,6-trimethylbenzoyl)-2,4,4-trimethylpentyl phosphine oxide, bis(2,6-dichlorobenzoyl)-2,4,4-trimethylpentyl phosphine oxide, 1-phenyl-2-hydroxy-2-methylpropan-1-one, 1-hydroxycyclohexylphenylketone, 1-(4-isopropylphenyl)-2-hydroxy-2-methylpropan-1-one, 1,2-diphenylethanedione, methylphenyl glyoxylate, 1-[4-(2-hydroxyethoxy)-phenyl]-2-hydroxy-2-methyl-1-propan-1-one, 2-hydroxy-1-{4-[4-(2-hydroxy-2-methyl-propionyl)-benzyl]phenyl}-2-methyl-propan-1-one, 2,2-dimethoxy-1,2-diphenylethan-1-one, 2-methyl-1-(4-methylthiophenyl)-2-morpholinopropan-1-one, 2-benzyl-2-dimethylamino-1-(4-morpholinophenyl)-butanone-1,2,4,6-trimethylbenzoyl-diphenyl-phosphine oxide, and bis(2,4,6-trimethylbenzoyl)-phenylphosphine oxide.

Among these, in the present invention, as the photoradical polymerization initiator, 1-hydroxycyclohexylphenylketone (available as Irgacure 184 (product name) from BASF), bis(2,4,6-trimethylbenzoyl)-phenylphosphine oxide (available as Irgacure 819 (product name) from BASF), 2,4,6-trimethylbenzoyl-diphenyl-phosphinoxide (available as Irgacure TPO (product name) from BASF Japan), 2,2,-dimethoxy-1,2-diphenylethan-1-one (available as Irgacure 651 (product name) from BASF), 1-[4-(2-hydroxyethoxy)-phenyl]-2-hydroxy-2-methyl-1-propan-1-one, or 2-methyl-1-(4-methylthiophenyl)-2-morpholinopropan-1-one can be preferably used.

In a case of containing a photoradical polymerization initiator, a content of the photoradical polymerization initiator in the above-described curable resin composition is preferably 0.01% to 5.0% by mass, more preferably 0.05% to 1.0% by mass, and still more preferably 0.05% to 0.5% by mass.

The curable resin composition according to the embodiment of the present invention may contain both the photoradical polymerization initiator and the thermal radical polymerization initiator. In this case, the total content of the photoradical polymerization initiator and the thermal radical polymerization initiator is preferably 0.01% to 5.0% by mass, more preferably 0.05% to 1.0% by mass, and still more preferably 0.05% to 0.5% by mass with respect to the total mass of the curable resin composition.

The curable resin composition containing the compound represented by General Formula (1) may include a polymer or a monomer other than the above-described components, a dispersant, a plasticizer, a heat stabilizer, a mold release agent, a solvent, or the like as long as the gist of the invention is maintained.

In a case of being used as a material for nanoimprint, it is preferable that the content of the inorganic particles in the curable resin composition is 30% by mass or less, and it is more preferable that the curable resin composition does not contain the inorganic particles.

From the viewpoint of improving handleability in a case of molding the cured product, particularly improving mold followability in a case of the nanoimprint to a high-quality cured product, a viscosity of the curable resin composition containing the compound represented by General Formula (1) is preferably 1 to 500 mPa·s, more preferably 1 to 400 mPa·s, and still more preferably 1 to 200 mPa·s.

The viscosity of the above-described curable resin composition is a viscosity under a condition of 25° C. and a shear rate of 10 s⁻¹, which is measured by a method described in Examples later. 1 mPa·s is 1 cP.

The curable resin composition according to the embodiment of the present invention can be used for producing a cured product which is required to have a high refractive index and excellent heat resistance.

Among these, since the curable resin composition according to the embodiment of the present invention has suppressed crystal precipitation, the curable resin composition according to the embodiment of the present invention can be preferably used as a material for nanoimprint to produce a cured product which achieves both high refractive index and excellent heat resistance.

### [Cured Product]

The cured product according to the embodiment of the present invention is a cured product of the above-described curable resin composition containing the compound represented by General Formula (1) described above.

The cured product according to the embodiment of the present invention is obtained by advancing a polymerization reaction of a monomer including the compound represented by General Formula (1) and curing the monomer. The cured product according to the embodiment of the present invention may contain an unreacted monomer (for example, the compound represented by General Formula (1)) and the like.

As described above, the cured product according to the embodiment of the present invention can exhibit a high refractive index and excellent heat resistance.

The refractive index of the cured product can be evaluated using a refractive index nD at a wavelength of 589 nm at 25° C.

The cured product according to the embodiment of the present invention can have a refractive index nD of 1.650 or more, preferably 1.670 or more, more preferably 1.680 or more, still more preferably 1.685 or more, and particularly preferably 1.690 or more. The upper limit value of the refractive index nD of the cured product according to the embodiment of the present invention is not particularly limited, but is practically 1.800 or less.

The refractive index nD is a value measured using an Abbe refractometer (for example, manufactured by Atago Co., Ltd., product name: multi-wavelength Abbe refractometer DR-M2 or DR-M4), and specifically, a sample (cured product) for measurement can be produced and measured according to the description of Examples later. In a case of forming the cured product, a heating step may be employed instead of an ultraviolet irradiating step described in Examples later, or both the heating step and the ultraviolet irradiating step may be employed. In addition, JIS (Japan Industrial Standards) B 7090:1999 optics and optical equipment-reference wavelengths (ISO (International Organization for Standardization) 7944:1998 Optics and optical instruments-Reference wavelengths) can be appropriately referred to.

A transmittance of the cured product according to the embodiment of the present invention over the entire visible light wavelength range of 360 to 830 nm is preferably 85% or more, more preferably 90% or more, and still more preferably 95% or more. The upper limit value of the transmittance of the cured product according to the embodiment of the present invention is not particularly limited, but is practically 99% or less.

The above-described transmittance of the cured product is a value of an external transmittance including surface reflection, which is measured using an ultraviolet-visible spectrophotometer (for example, UV-2600 (product name), manufactured by Shimadzu Corporation) for a cured product having a thickness of 1 mm.

### [Method for Manufacturing Cured Product]

The cured product according to the embodiment of the present invention can be manufactured by a method including at least one of a step of photocuring the above-described curable resin composition or a step of thermosetting the above-described curable resin composition. It is preferable that the above-described photoradical polymerization initiator is contained in the curable resin composition in a case of photocuring, or the above-described thermal radical polymerization initiator is contained in the curable resin composition in a case of heat-curing.

As for the photocuring conditions, a description of light irradiation in a diffractive optical element described later can be preferably applied.

In the heat curing, the heating temperature can be, for example, 150° C. or higher, and is preferably 160° C. to 270° C., more preferably 165° C. to 250° C., and still more preferably 170° C. to 230° C. During heating, pressurization may be performed together with the heating.

The pressure in a case of pressurization is preferably 0.098 MPa to 9.8 MPa, more preferably 0.294 MPa to 4.9 MPa, and still more preferably 0.294 MPa to 2.94 MPa.

The heat-curing time is preferably 30 to 1000 seconds, more preferably 30 to 500 seconds, and still more preferably 60 to 300 seconds. The atmosphere during the heat curing (thermopolymerization) is preferably an atmosphere replaced with air or an inert gas, and more preferably an atmosphere in which air is replaced with nitrogen until the oxygen concentration is 1% or less.

In addition, the method for manufacturing the cured product according to the embodiment of the present invention preferably includes a step of forming a semi-cured product by irradiating the curable resin composition with light or heating the curable resin composition; and a step of forming a cured product by irradiating the obtained semi-cured product with light or heating the obtained semi-cured product. The “forming” is used to include obtaining a cured product in addition to the actual forming.

As each of the “step of forming a semi-cured product”, the “step of forming a cured product”, and the “semi-cured product”, the description of the “step of forming a semi-cured product”, the “step of forming a cured product”, and the “semi-cured product” in [0106] to [0117], [0118] to [0124], and [0125] of WO2019/044863A can be adopted as they are.

### [Use of Cured Product]

The cured product according to the embodiment of the present invention can be used for various applications because it exhibits high refractive index and excellent heat resistance, and can be preferably used for a diffractive optical element.

### [Diffractive Optical Element]

The diffractive optical element according to an embodiment of the present invention is a diffractive optical element including a surface which has a diffraction grating shape and is formed of the cured product according to the embodiment of the present invention, and is formed by curing the curable resin composition according to the embodiment of the present invention.

The diffractive optical element according to the embodiment of the present invention preferably has a maximum thickness of 0.05 μm to 100 μm. The maximum thickness is more preferably 0.1 μm to 50 μm and still more preferably 0.2 μm to 30 μm. In addition, a level difference (lattice thickness) of the diffraction grating shape (periodic structure) included in the diffractive optical element is preferably 0.05 μm to 50 μm and more preferably 0.1 μm to μm. Furthermore, it is sufficient that a pitch of the diffraction grating shape included in the diffractive optical element is in a range of 0.05 μm to 1 mm, and it is preferable that the pitch is changed according to the required optical aberration in the same diffractive optical element. In particular, in a case where the diffractive optical element according to the embodiment of the present invention is used as a diffractive optical element for a waveguide, the level difference of the diffraction grating shape is preferably 0.05 μm to 100 m, and the pitch of the diffractive grating shape is preferably 0.05 μm to 100 μm.

The diffractive optical element can be manufactured according to, for example, the following procedure.

The curable resin composition is sandwiched between a surface of a mold, which is processed into a diffraction grating shape, and a transparent substrate. Thereafter, the curable resin composition may be pressurized and stretched to a desired range. In the sandwiched state, the curable resin composition is irradiated with light from the transparent substrate side to cure the curable resin composition. Thereafter, the cured product is released from a mold such as a metal mold. After the mold release, the cured product may be further irradiated with light from the side opposite to the transparent substrate side.

Examples of the transparent substrate include a flat glass, and a flat transparent resin (such as (meth)acrylic resin, polycarbonate resin, and polyethylene terephthalate).

The transparent substrate used in the above-described production may be included in the diffractive optical element as it is, or may be peeled off.

As the light used for the light irradiation curing the curable resin composition, ultraviolet rays or visible rays are preferable and ultraviolet rays are more preferable. For example, a metal halide lamp, a low pressure mercury lamp, a high pressure mercury lamp, an ultra-high pressure mercury lamp, a germicidal lamp, a xenon lamp, a light emitting diode (LED) light source lamp, and the like are suitably used. The illuminance of ultraviolet light used for the light irradiation curing the curable resin composition is preferably 1 to 100 mW/cm², more preferably 1 to 75 mW/cm², and still more preferably 5 to 50 mW/cm². The curable resin composition may be irradiated with ultraviolet light having different illuminance multiple times. The exposure amount of ultraviolet light is preferably 0.4 to 10 J/cm², more preferably 0.5 to 5 J/cm², and still more preferably 1 to 3 J/cm². The atmosphere during the light irradiation is preferably an atmosphere replaced with air or an inert gas, and more preferably an atmosphere in which air is replaced with nitrogen until the oxygen concentration is 1% or less.

The diffractive optical element according to the embodiment of the present invention can be suitably used as a diffractive optical element for a waveguide in AR glasses.

The diffractive optical element for a waveguide in the AR glasses can be usually manufactured by nanoimprint.

Using the curable resin composition according to the embodiment of the present invention as a material for nanoimprint, the material is pressed in a mold having a desired pattern in a nanometer unit in a state of being embossed, and then photo-cured or thermally cured to produce the cured product according to the embodiment of the present invention, to which the pattern of the mold is transferred. Thereafter, the cured product is released from the mold to produce the diffraction grating shape.

As the conditions for the photocuring or thermal curing, the description in the manufacturing method of the cured product and the description in the manufacturing of the diffractive optical element above can be applied.

For the points other than the above point, the description of the general nanoimprint can be adopted without particular limitation, and for example, a nanoimprint technology handbook (edited by the Japan Society of Applied Physics and Nanoimprint Technology Research Association, published by Ohmsha, Dec. 1, 2019) can be referred to.

### [Augmented Reality (AR) Glasses]

The diffractive optical element according to the embodiment of the present invention can be used as a light guide plate of an AR glass.

As the AR glasses, the diffractive optical element according to the embodiment of the present invention can be used as a light guide plate which guides an image output from a microdisplay to eyes of a wearer, and a configuration of the AR glasses in general can be adopted without particular limitation.

An input unit diffraction element (In-coupling Grating) and an output unit diffraction element (Out-coupling Grating) are formed on the light guide plate, and the image incident from the display is diffracted by the input unit diffraction element and advances in an in-plane direction of the light guide plate. The image guided through the inside of the light guide plate to the front of the eyes of the wearer by the total reflection is guided to the eyes of the wearer by the output unit diffraction element, by changing a course to the outside of the light guide plate. The diffractive optical element according to the embodiment of the present invention can also be used for at least one of the input unit diffraction element or the output unit diffraction element.

Since the AR glasses according to the embodiment of the present invention include the diffractive optical element exhibiting a high refractive index and excellent heat resistance, the AR glasses have a wide viewing angle and excellent durability.

### EXAMPLES

Hereinafter, the present invention will be described in more detail based on Examples. The materials, amounts used, proportions, treatment details, treatment procedures, and the like described in the following examples can be appropriately modified as long as the gist of the invention is maintained. Therefore, the scope of the present invention should not be construed as being limited to the following specific examples.

All steps from the preparation of the curable resin composition to the test of the cured product were carried out in an environment where a yellow lamp was used as lighting.

### Synthesis Examples

The compound represented by General Formula (1) was synthesized as follows.

### Synthesis Example 1: Synthesis of Compound (A-9)

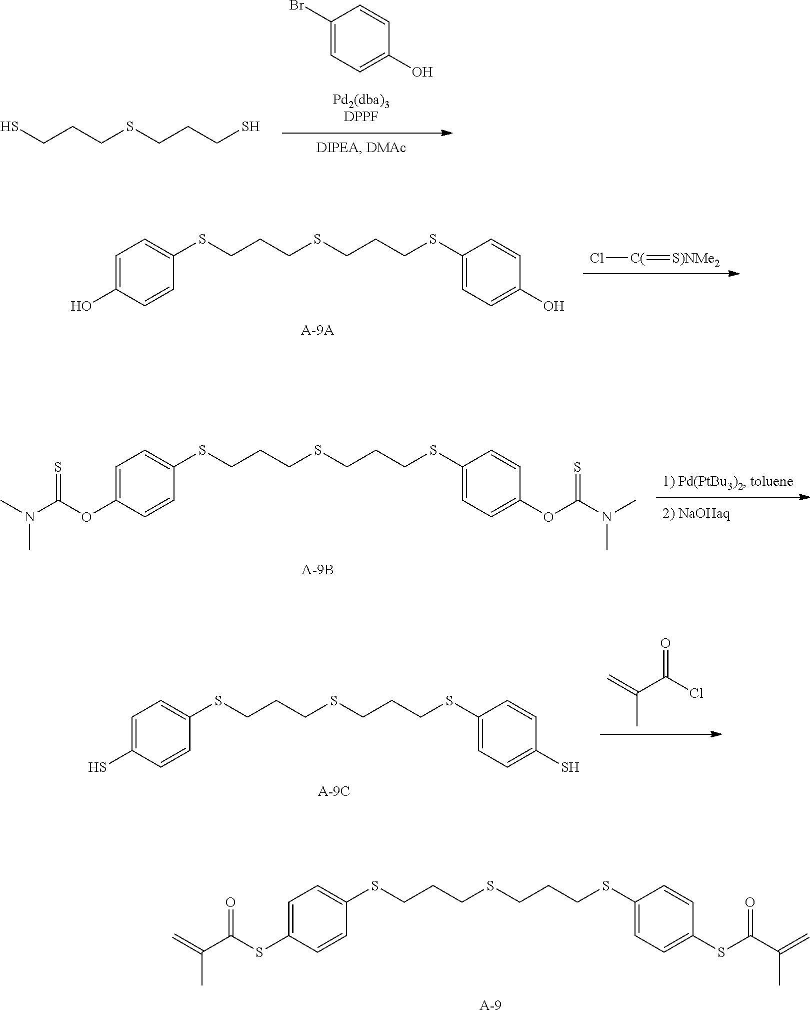

### <Synthesis of Compound (A-9A)>

11.9 g (0.065 mol) of bis(3-mercaptopropyl)sulfide synthesized with reference to the method described in “Synthesis, 1981, 6, 457”, 98 mL of N,N-dimethylacetamide (DMAc), 33.7 g (0.261 mol) of N,N-diisopropylethylamine (DIPEA), and 22.6 g (0.131 mol) of p-bromophenol were mixed and then subjected to nitrogen replacement, 2.99 g (3.26 mmol) of tris(dibenzylideneacetone)dipalladium (Pd₂(dba)₃) and 3.62 g (6.53 mmol) of diphenylphosphinophenylferrocene (DPPF) were added thereto, and the mixture was heated such that the internal temperature (liquid temperature) was 80° C. After stirring for 2 hours, 300 mL of ethyl acetate and 300 mL of a 1 N hydrochloric acid were added thereto, and the mixture was stirred, washed, and liquid-separated. Next, 300 mL of water was added thereto and stirred, and then the mixture was washed and liquid-separated. An oily composition was obtained by dehydration with magnesium sulfate, filtration, concentration, and then purified by column chromatography to obtain 13.2 g of a compound (A-9A) (yield: 56%).

### <Synthesis of Compound (A-9B)>

3.25 g (8.87 mmol) of the compound (A-9A), 2.41 g (19.5 mmol) of dimethylthiocarbamoyl chloride, 2.15 g (21.3 mmol) of triethylamine, and 22 mL of N,N-dimethylacetamide were mixed with each other, and heated while maintaining an internal temperature (liquid temperature) at 70° C. After stirring for 24 hours, 30 mL of ethyl acetate and 30 mL of a 1 N hydrochloric acid were added thereto, and the mixture was stirred, washed, and liquid-separated. Next, 150 mL of a 5% sodium hydrogen carbonate aqueous solution was added thereto, and the mixture was stirred, washed, and liquid-separated. An oily composition was obtained by dehydration with magnesium sulfate, filtration, concentration, and then purified by column chromatography to obtain 2.15 g of a compound (A-9B) (yield: 45%).

### <Synthesis of Compound (A-9C)>

3 g (5.54 mmol) of the compound (A-9B) and 20 mL of toluene were mixed and then subjected to nitrogen replacement, 0.142 g (0.277 mmol) of bis(tri-t-butylphosphine)palladium (Pd(PtBu₃)₂) was added thereto, and the mixture was heated such that the internal temperature (liquid temperature) was 70° C. After stirring for 24 hours, 30 mL of a 1 N sodium hydroxide aqueous solution was added thereto, and the mixture was stirred at 50° C. for 24 hours. 3 mL of concentrated hydrochloric acid was added thereto, and then the mixture washed and liquid-separated. Next, 30 mL of water was added thereto and stirred, and then the mixture was washed and liquid-separated. An oily composition was obtained by dehydration with magnesium sulfate, filtration, concentration, and then purified by column chromatography to obtain 0.95 g of a compound (A-9C) (yield: 43%).

### <Synthesis of Compound (A-9)>

3 g (7.53 mmol) of the compound (A-9C) and 15 mL of N,N-dimethylacetamide were mixed and then cooled to 0° C., 2.33 g (18.1 mmol) of N,N-diisopropylethylamine and 1.65 g (15.8 mmol) of methacryloyl chloride were added dropwise thereto, and the mixture was heated such that the internal temperature (liquid temperature) was 25° C. After stirring for 2 hours, 50 mL of ethyl acetate and 50 mL of a 1 N hydrochloric acid were added thereto, and the mixture was stirred, washed, and liquid-separated. Next, 50 mL of a 5% sodium hydrogen carbonate aqueous solution was added thereto, and the mixture was stirred, washed, and liquid-separated. An oily composition was obtained by dehydration with magnesium sulfate, filtration, concentration, and then purified by column chromatography to obtain 2.221 g of a compound (A-9) (yield: 55%).

¹H-NMR (300 MHz, CDCl₃): δ (ppm) 2.0 to 2.2 (m, 10H), 2.48 (t, 4H), 2.94 (t, 4H), 5.60 (s, 2H), 6.10 (s, 2H), 7.2 to 7.4 (m, 8H)

### Synthesis Example 2: Synthesis of Compound (A-10)

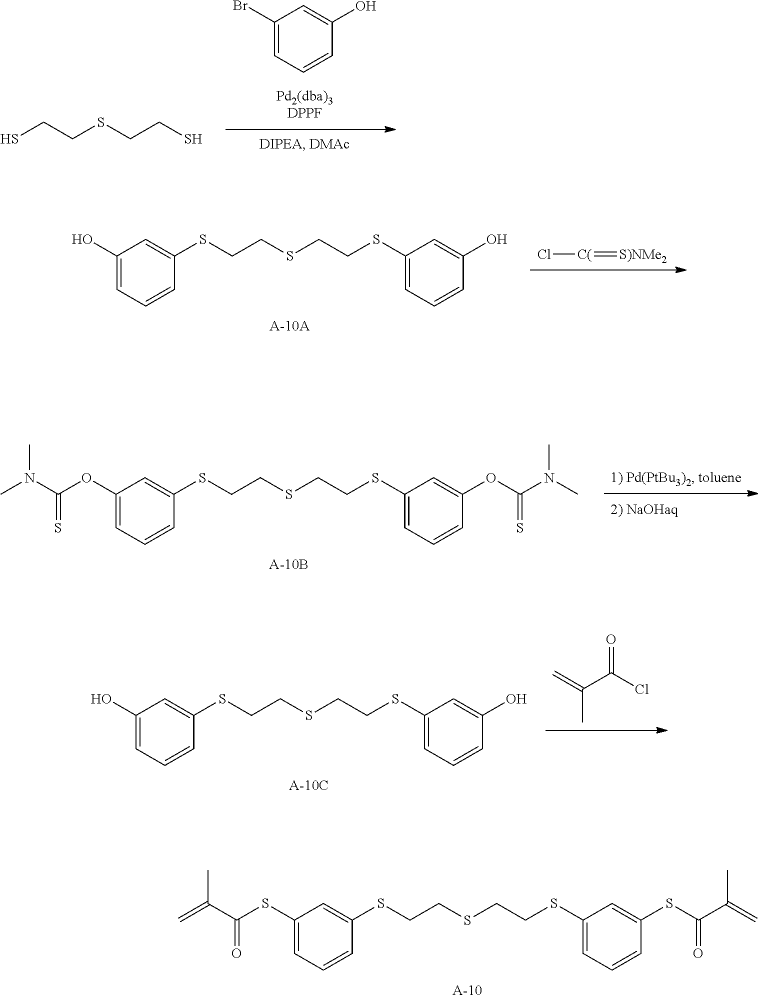

### <Synthesis of Compound (A-10A)>

A compound (A-10A) was synthesized in the same manner as in the synthesis of the compound (A-9A) (yield: 57%), except that the (3-mercaptopropyl)sulfide was changed to (2-mercaptoethyl)sulfide and the p-bromophenol was changed to m-bromophenol.

### <Synthesis of Compound (A-10B)>

A compound (A-10B) was synthesized in the same manner as in the synthesis of the compound (A-9B) (yield: 48%), except that the compound (A-9A) was changed to the compound (A-10A).

### <Synthesis of Compound (A-10C)>

A compound (A-10C) was synthesized in the same manner as in the synthesis of the compound (A-9C) (yield: 47%), except that the compound (A-9B) was changed to the compound (A-10B).

### <Synthesis of Compound (A-10)>

A compound (A-10) was synthesized in the same manner as in the synthesis of the compound (A-9) (yield: 50%), except that the compound (A-9C) was changed to the compound (A-10C).

¹H-NMR (300 MHz, CDCl₃): δ (ppm) 2.05 (s, 6H), 2.6 to 2.8 (m, 4H), 3.0 to 3.2 (m, 4H), 5.60 (s, 2H), 6.10 (s, 2H), 7.0 to 7.3 (m, 8H)

### Synthesis Example 3: Synthesis of Compound (B-1)

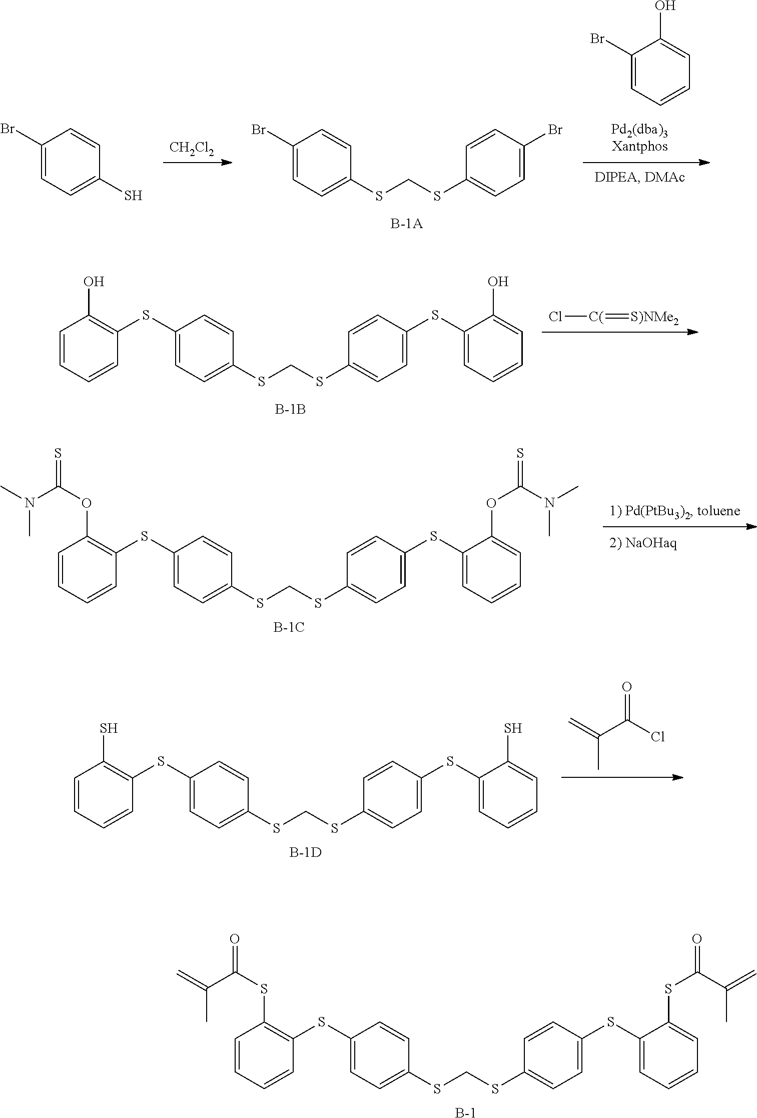

### <Synthesis of Compound (B-1A)>

20.0 g (0.106 mol) of 4-bromobenzenethiol, 100 mL of dichloromethane, and 14.4 g (0.111 mol) of N,N-diisopropylethylamine (DIPEA) were mixed and heated at an internal temperature (liquid temperature) of 25° C. After stirring for 1 hour, 400 mL of ethyl acetate and 200 mL of a 1 N hydrochloric acid were added thereto, and the mixture was stirred, washed, and liquid-separated. Next, 200 mL of a 5% sodium hydrogen carbonate aqueous solution was added thereto, and the mixture was stirred, washed, and liquid-separated. The magnesium sulfate was removed by dehydration and filtration, the filtrate was concentrated, and then 15 mL of ethyl acetate and 40 mL of hexane were added thereto for recrystallization, thereby obtaining 17.1 g of a compound (B-1A) (yield: 83%).

### <Synthesis of Compound (B-1B)>

10.0 g (0.0256 mol) of the compound (B-1A), 38 mL of N,N-dimethylacetamide (DMAc), 13.9 g (0.108 mol) of N,N-diisopropylethylamine (DIPEA), and 6.79 g (0.0538 mol) of o-thiophenol were mixed and then subjected to nitrogen replacement, 1.17 g (1.28 mmol) of tris(dibenzylideneacetone)dipalladium (Pd₂(dba)₃) and 1.48 g (2.56 mmol) of 4,5-bis(diphenylphosphino)-9,9-dimethylxanthene (Xantphos) were added thereto, and the mixture was heated such that the internal temperature (liquid temperature) was 120° C. After stirring for 2 hours, 300 mL of ethyl acetate and 200 mL of a 1 N hydrochloric acid were added thereto, and the mixture was stirred, washed, and liquid-separated. Next, 200 mL of a 5% sodium hydrogen carbonate aqueous solution was added thereto, and the mixture was stirred, washed, and liquid-separated. An oily composition was obtained by dehydration with magnesium sulfate, filtration, concentration, and then purified by column chromatography to obtain 11.1 g of a compound (B-1B) (yield: 90%).

### <Synthesis of Compound (B-1C)>

3.25 g (6.76 mmol) of the compound (B-1B), 1.84 g (14.9 mmol) of dimethylthiocarbamoyl chloride, 1.64 g (16.2 mmol) of triethylamine, and 17 mL of N,N-dimethylacetamide were mixed with each other, and heated while maintaining an internal temperature (liquid temperature) at 70° C. After stirring for 24 hours, 30 mL of ethyl acetate and 30 mL of a 1 N hydrochloric acid were added thereto, and the mixture was stirred, washed, and liquid-separated. Next, 15 mL of a 5% sodium hydrogen carbonate aqueous solution was added thereto, and the mixture was stirred, washed, and liquid-separated. An oily composition was obtained by dehydration with magnesium sulfate, filtration, concentration, and then purified by column chromatography to obtain 1.77 g of a compound (B-1C) (yield: 40%).

### <Synthesis of Compound (B-1D)>

3 g (4.58 mmol) of the compound (B-1C) and 15 mL of toluene were mixed and then subjected to nitrogen replacement, 0.117 g (0.229 mmol) of bis(tri-t-butylphosphine)palladium (Pd(PtBu₃)₂) was added thereto, and the mixture was heated such that the internal temperature (liquid temperature) was 70° C. After stirring for 24 hours, 15 mL of a 1 N sodium hydroxide aqueous solution was added thereto, and the mixture was stirred at 50° C. for 8 hours. 3 mL of concentrated hydrochloric acid was added thereto, and then the mixture washed and liquid-separated. Next, 30 mL of water was added thereto and stirred, and then the mixture was washed and liquid-separated. An oily composition was obtained by dehydration with magnesium sulfate, filtration, concentration, and then purified by column chromatography to obtain 1.05 g of a compound (B-1D) (yield: 45%).

### <Synthesis of Compound (B-1)>

3 g (5.85 mmol) of the compound (B-1D) and 15 mL of N,N-dimethylacetamide were mixed and then cooled to 0° C., 1.82 g (12.3 mmol) of N,N-diisopropylethylamine and 1.28 g (12.3 mmol) of methacryloyl chloride were added dropwise thereto, and the mixture was heated such that the internal temperature (liquid temperature) was 25° C. After stirring for 2 hours, 50 mL of ethyl acetate and 50 mL of a 1 N hydrochloric acid were added thereto, and the mixture was stirred, washed, and liquid-separated. Next, 50 mL of a 5% sodium hydrogen carbonate aqueous solution was added thereto, and the mixture was stirred, washed, and liquid-separated. An oily composition was obtained by dehydration with magnesium sulfate, filtration, concentration, and then purified by column chromatography to obtain 2.0 g of a compound (B-1) (yield: 53%).

¹H-NMR (300 MHz, DMSO-d₆): δ (ppm) 2.02 (s, 6H), 4.65 (s, 2H), 5.65 (s, 2H), 6.00 (s, 2H), 7.0 to 7.3 (m, 16H)

### Synthesis Example 4: Synthesis of Compound (B-4)

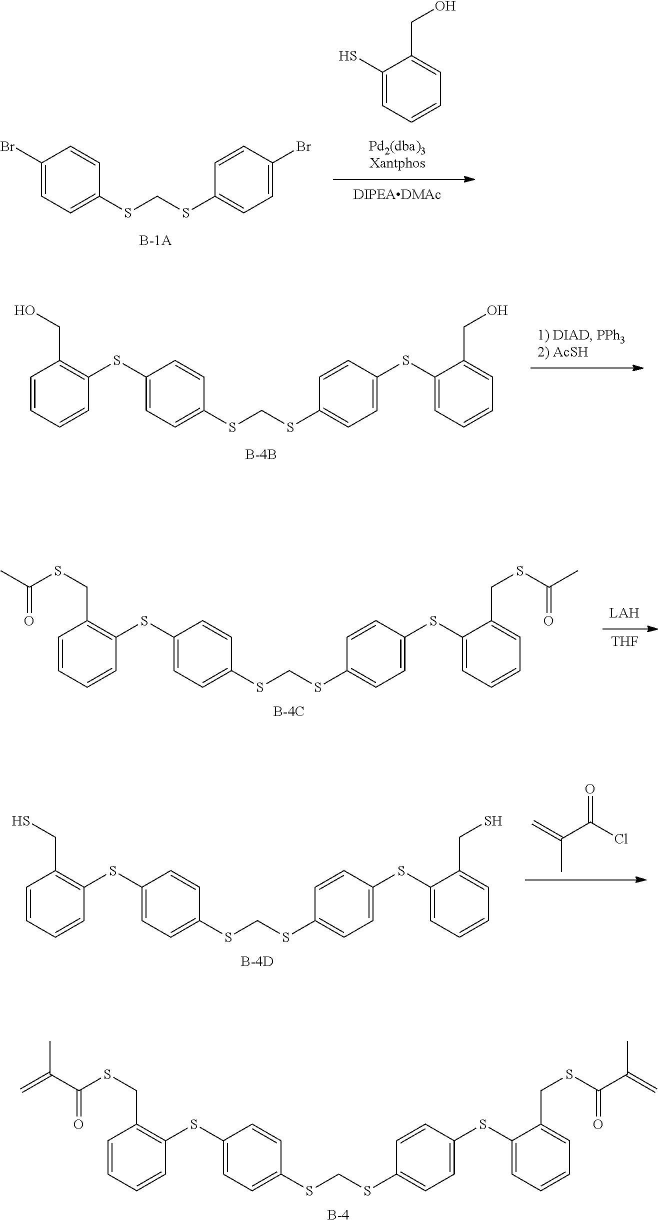

### <Synthesis of Compound (B-4B)>

10.0 g (0.0256 mol) of the compound (B-1A), 38 mL of N,N-dimethylacetamide (DMAc), 13.9 g (0.108 mol) of N,N-diisopropylethylamine (DIPEA), and 7.55 g (0.0538 mol) of o-thiobenzyl alcohol were mixed and then subjected to nitrogen replacement, 1.17 g (1.28 mmol) of tris(dibenzylideneacetone)dipalladium (Pd₂(dba)₃) and 1.48 g (2.56 mmol) of 4,5-bis(diphenylphosphino)-9,9-dimethylxanthene (Xantphos) were added thereto, and the mixture was heated such that the internal temperature (liquid temperature) was 120° C. After stirring for 2 hours, 300 mL of ethyl acetate and 200 mL of a 1 N hydrochloric acid were added thereto, and the mixture was stirred, washed, and liquid-separated. Next, 200 mL of a 5% sodium hydrogen carbonate aqueous solution was added thereto, and the mixture was stirred, washed, and liquid-separated. An oily composition was obtained by dehydration with magnesium sulfate, filtration, concentration, and then purified by column chromatography to obtain 11.7 g of a compound (B-4B) (yield: 91%).

### <Synthesis of Compound (B-4C)>

7.37 g (2.81 mmol) of triphenylphosphine (PPh₃) and 65 mL of tetrahydrofuran (THF) were mixed and then subjected to nitrogen replacement, and the mixture was cooled to an internal temperature (liquid temperature) of 0° C. 5.68 g (14.9 mmol) of diisopropyl azodicarboxylate (DIAD) was added dropwise thereto, the mixture was stirred at 0° C. for 30 minutes, and then a mixed solution of 3.25 g (6.39 mmol) of the compound (B-4B), 1.36 g (1.79 mmol) of thioacetic acid (AcSH), and 65 mL of tetrahydrofuran (THF) was added dropwise thereto such that the liquid temperature did not exceed 7° C., and the mixture was stirred for 1 hour. After heating to 25° C., 200 mL of ethyl acetate and 150 mL of a 5% sodium hydrogen carbonate aqueous solution were added thereto, and the mixture was stirred, washed, and liquid-separated. An oily composition was obtained by dehydration with magnesium sulfate, filtration, concentration, and then purified by column chromatography to obtain 2.79 g of a compound (B-4C) (yield: 70%).

### <Synthesis of Compound (B-4D)>

0.273 g (7.20 mmol) of lithium aluminum hydride (LAH) and 54 mL of tetrahydrofuran (THF) were mixed and then subjected to nitrogen replacement, and the mixture was cooled to an internal temperature (liquid temperature) of 0° C. A mixed solution of 3 g (4.80 mmol) of the compound (B-4C) and 34 mL of tetrahydrofuran (THF) was added dropwise thereto such that the liquid temperature did not exceed 7° C., and the mixture was heated to an internal temperature (liquid temperature) of 25° C. After stirring for 1 hour, the mixture was cooled to 0° C., 60 mL of ethyl acetate, 6 g of concentrated hydrochloric acid, and 60 mL of water were each added dropwise thereto in this order, and then the mixture was washed and liquid-separated. Next, 60 mL of water was added thereto and stirred, and then the mixture was washed and liquid-separated. An oily composition was obtained by dehydration with magnesium sulfate, filtration, concentration, and then purified by column chromatography to obtain 2.02 g of a compound (B-4D) (yield: 78%).

### <Synthesis of Compound (B-4)>

A compound (B-4) was synthesized in the same manner as in the synthesis of the compound (B-1) (yield: 42%), except that the compound (B-1D) was changed to the compound (B-4D).

¹H-NMR (300 MHz, DMSO-d₆): δ (ppm) 2.01 (s, 6H), 4.46 (s, 4H), 4.65 (s, 2H), 5.60 (s, 2H), 6.10 (s, 2H), 7.1 to 7.2 (m, 4H), 7.3 to 7.6 (m, 12H)

### Synthesis Example 5: Synthesis of Compound (B-10)

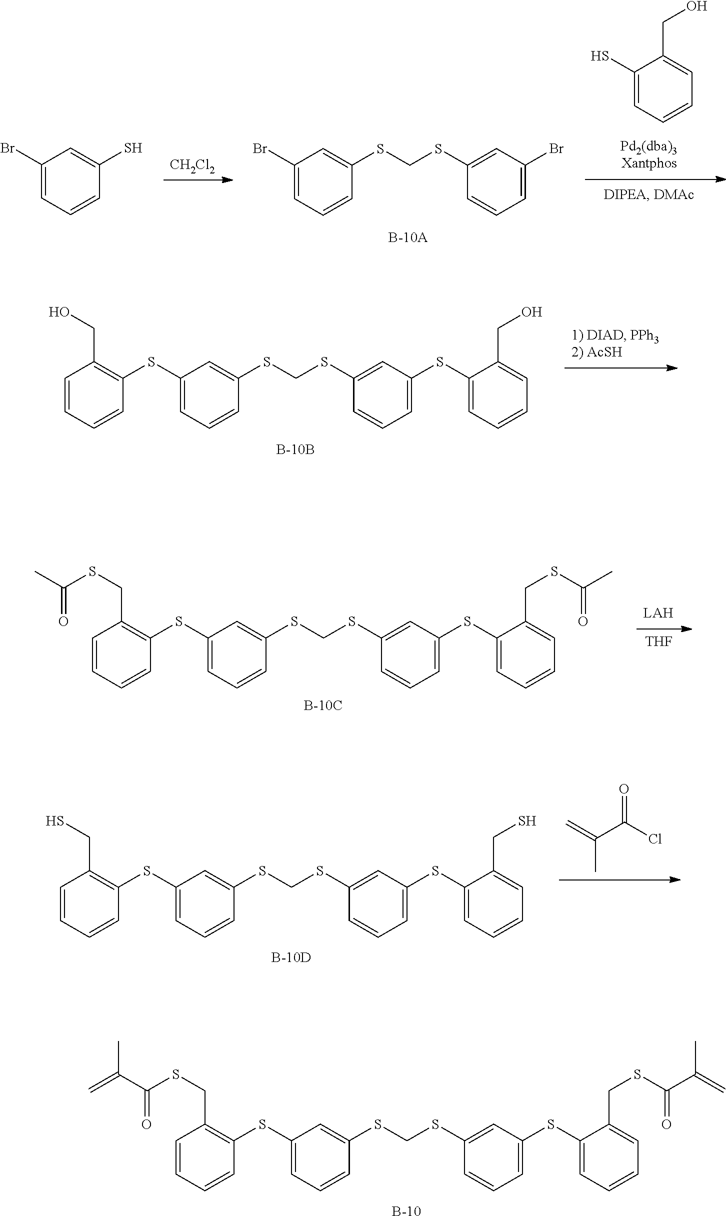

### <Synthesis of Compound (B-10A)>

A compound (B-10A) was synthesized in the same manner as in the synthesis of the compound (B-1A) (yield: 97%), except that the p-bromobenzenethiol was changed to m-bromobenzenethiol.

### <Synthesis of Compound (B-10B)>

A compound (B-10B) was synthesized in the same manner as in the synthesis of the compound (B-4B) (yield: 88%), except that the compound (B-1A) was changed to the compound (B-10A).

### <Synthesis of Compound (B-10C)>

A compound (B-10C) was synthesized in the same manner as in the synthesis of the compound (B-4C) (yield: 65%), except that the compound (B-4B) was changed to the compound (B-10B).

### <Synthesis of Compound (B-10D)>

A compound (B-10D) was synthesized in the same manner as in the synthesis of the compound (B-4D) (yield: 75%), except that the compound (B-4C) was changed to the compound (B-10C).

### <Synthesis of Compound (B-10)>

A compound (B-10) was synthesized in the same manner as in the synthesis of the compound (B-1) (yield: 46%), except that the compound (B-1D) was changed to the compound (B-10D).

¹H-NMR (300 MHz, DMSO-d₆): δ (ppm) 2.01 (s, 6H), 4.46 (s, 4H), 4.65 (s, 2H), 5.60 (s, 2H), 6.10 (s, 2H), 7.1 to 7.6 (m, 16H)

### Synthesis Example 6: Synthesis of Compound (A-12)

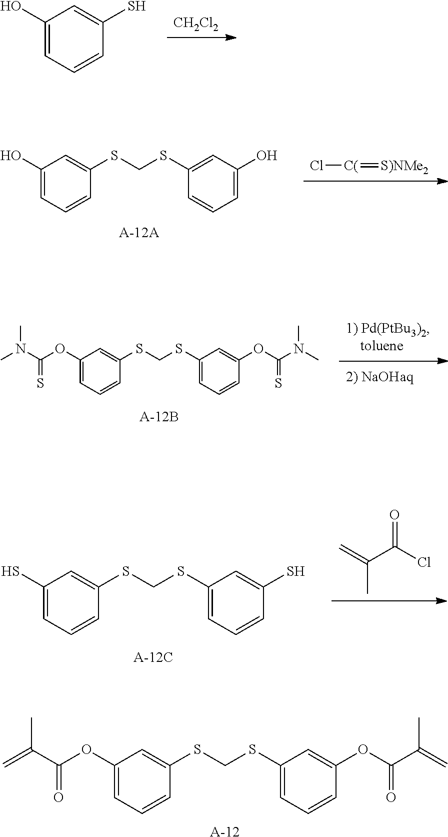

### <Synthesis of Compound (A-12A)>

A compound (A-12A) was synthesized in the same manner as in the synthesis of the compound (B-1A) (yield: 85%), except that the p-bromobenzenethiol was changed to m-mercaptophenol.

### <Synthesis of Compound (A-12B)>

A compound (A-12B) was synthesized in the same manner as in the synthesis of the compound (B-1C) (yield: 75%), except that the compound (B-1B) was changed to the compound (A-12A).

### <Synthesis of Compound (A-12C)>

A compound (A-12C) was synthesized in the same manner as in the synthesis of the compound (B-1D) (yield: 80%), except that the compound (B-1C) was changed to the compound (A-12B).

### <Synthesis of Compound (A-12)>

A compound (A-12) was synthesized in the same manner as in the synthesis of the compound (B-1) (yield: 42%), except that the compound (B-1D) was changed to the compound (A-12C).

¹H-NMR (300 MHz, DMSO-d₆): δ (ppm) 2.05 (s, 6H), 4.68 (s, 2H), 5.65 (s, 2H), 6.01 (s, 2H), 7.0 to 7.4 (m, 8H)

### Example 1: Preparation of Curable Resin Composition

A curable resin composition was prepared by mixing a compound represented by General Formula (1) or a comparative compound, a dilution monomer, and a photopolymerization initiator so as to have a composition shown in Table 1 below, and homogeneously stirring the mixture while heating the mixture to 40° C.

Curable resin compositions Nos. 101 to 108 are Examples, and curable resin compositions Nos. c11 to c13 are Comparative Examples.

In a case where a comparative compound Z-1 was used, as shown in the curable resin composition No. c12, the evaluation of crystal precipitation of the composition, which will be described later, could be set to “A” only by suppressing a blending amount of the compound Z-1 to 50% by mass or less.

Viscosites of the curable resin compositions Nos. 101 to 108 were measured using a reometer (product name: RS600) manufactured by HAAKE under the conditions of 25° C. and a shear rate of 10 s⁻¹, and were all in a range of 50 to 300 mPa·s.

### Example 2: Production of Cured Product

The curable resin composition prepared above was sandwiched between glass plates subjected to a hydrophobization treatment, such that a film thickness of a cured product was 150 m, and the curable resin composition was irradiated with ultraviolet (UV) light under an atmosphere in which the oxygen concentration was 1% or less using a UV irradiation device (EXECURE 3000 (product name), manufactured by HOYA CANDEO OPTRONICS CORPORATION) in an atmosphere replaced with nitrogen (N₂) and under conditions of an integrated light amount 1.2 J/cm² and an illuminance 5 mW/cm², thereby producing a cured product.

### [Evaluation 1] Refractive Index of Cured Product

A refractive index nD of the obtained cured product at a wavelength of 589 nm was measured under a condition of 25° C. using a multi-wavelength Abbe refractometer DR-M2 or DR-M4 (product name, manufactured by Atago Co., Ltd.), and evaluated according to the following standard. The results are summarized in Table 1.

### —Evaluation Standard for Refractive Index of Cured Product—

$$A 3 : 1.69 \leq nD \\ A 2 : 1.685 \leq nD < 1.69 \\ A 1 : 1.68 \leq nD < 1.685 \\ B : 1.67 \leq nD < 1.68 \\ C : 1.65 \leq nD < 1.67 \\ D : nD < 1.65$$

### [Evaluation 2] Crystal Precipitation of Composition

The above-described curable resin composition prepared by homogeneously stirring while raising the temperature to 40° C. was stored at 25° C. under light shielding conditions, and it was visually observed whether or not crystal precipitation was observed with the passage of time, and the crystal precipitation of the composition was evaluated according to the following standard. The point in time at which the temperature of the curable resin composition was changed from 40° C. to 25° C. was set as the observation start point. The results are summarized in Table 1.

### —Evaluation Standard for Crystal Precipitation of Composition—

A: no crystal was precipitated even after 48 hours, and the solution remained liquid.
B: no crystal was precipitated even after 24 hours, and crystals were precipitated in less than 48 hours.
C: crystals were precipitated in less than 24 hours.

### [Evaluation 3] Heat Resistance of Cured Product

10 mg of the curable resin composition prepared above was placed on a mold having a diameter of 25 mm, a glass having a diameter of 30 mm was placed thereon, and UV (ultraviolet) irradiation was performed in the same manner as in the production of the cured product of Example 2 described above. Thereafter, the mold was removed to produce a glass-resin composite sample (cured product) having a thickness of 20 m.

All ten produced samples were stored at 150° C. for 120 hours, and the presence or absence of crack occurrence was visually observed, and the heat resistance was evaluated according to the following standard. The results are summarized in Table 1.

### —Evaluation Standard for Heat Resistance of Cured Product—

A: among all the samples, the number of samples in which cracks occurred was 0 or 1.
B: among all the samples, the number of samples in which cracks occurred was 2 or 3.
C: among all the samples, the number of samples in which cracks occurred was 4 or 5.
D: among all the samples, the number of samples in which cracks occurred was 6 or more.

TABLE 1

| Curable resin composition No. | | 101 | 102 | 103 | 104 | 105 | 106 |
|---|---|---|---|---|---|---|---|
| Compound represented | Type | A-9 | A-10 | B-1 | B-4 | B-10 | A-12 |
| by General Formula | Blending | 80 wt % | 80 wt % | 80 wt % | 80 wt % | 80 wt % | 80 wt % |
| (1) or comparative | amount | | | | | | |
| compound | | | | | | | |
| Dilution monomer: | Blending | 19.7 wt % | 19.7 wt % | 19.7 wt % | 19.7 wt % | 19.7 wt % | 19.7 wt % |
| M-1 | amount | | | | | | |
| Photopolymerization | Blending | 0.30 wt % | 0.30 wt % | 0.30 wt % | 0.30 wt % | 0.30 wt % | 0.30 wt % |
| initiator; IrgTPO | amount | | | | | | |

| Refractive index nD of cured product | | B | A1 | A2 | A2 | A2 | A1 |
|---|---|---|---|---|---|---|---|
| Crystal precipitation of composition | | A | A | A | A | A | A |
| Heat resistance of cured product | | A | A | A | A | A | A |
| | Curable resin composition No. | | 107 | 108 | c11 | c12 | c13 |
| | Compound represented | Type | A-10 | B-10 | Z-1 | Z-1 | Z-2 |
| | by General Formula | Blending | 99.7 wt % | 99.7 wt % | 80 wt % | 50 wt % | 80 wt % |
| | (1) or comparative | amount | | | | | |
| | compound | | | | | | |
| | Dilution monomer: | Blending | — | — | 19.7 wt % | 49.7 wt % | 19.7 wt % |
| | M-1 | amount | | | | | |
| | Photopolymerization | Blending | 0.30 wt % | 0.30 wt % | 0.30 wt % | 0.30 wt % | 0.30 wt % |
| | initiator; IrgTPO | amount | | | | | |

| | | | | | |
|---|---|---|---|---|---|
| Refractive index nD of cured product | A3 | A3 | A1 | D | B |
| Crystal precipitation of composition | A | A | C | A | A |
| Heat resistance of cured product | A | A | A | A | C |

### <Note to Table>

Each component in the tables is as follows. The blending amount wt % of each component described in the column of each component is % by mass, and “-” indicates that the component was not contained.

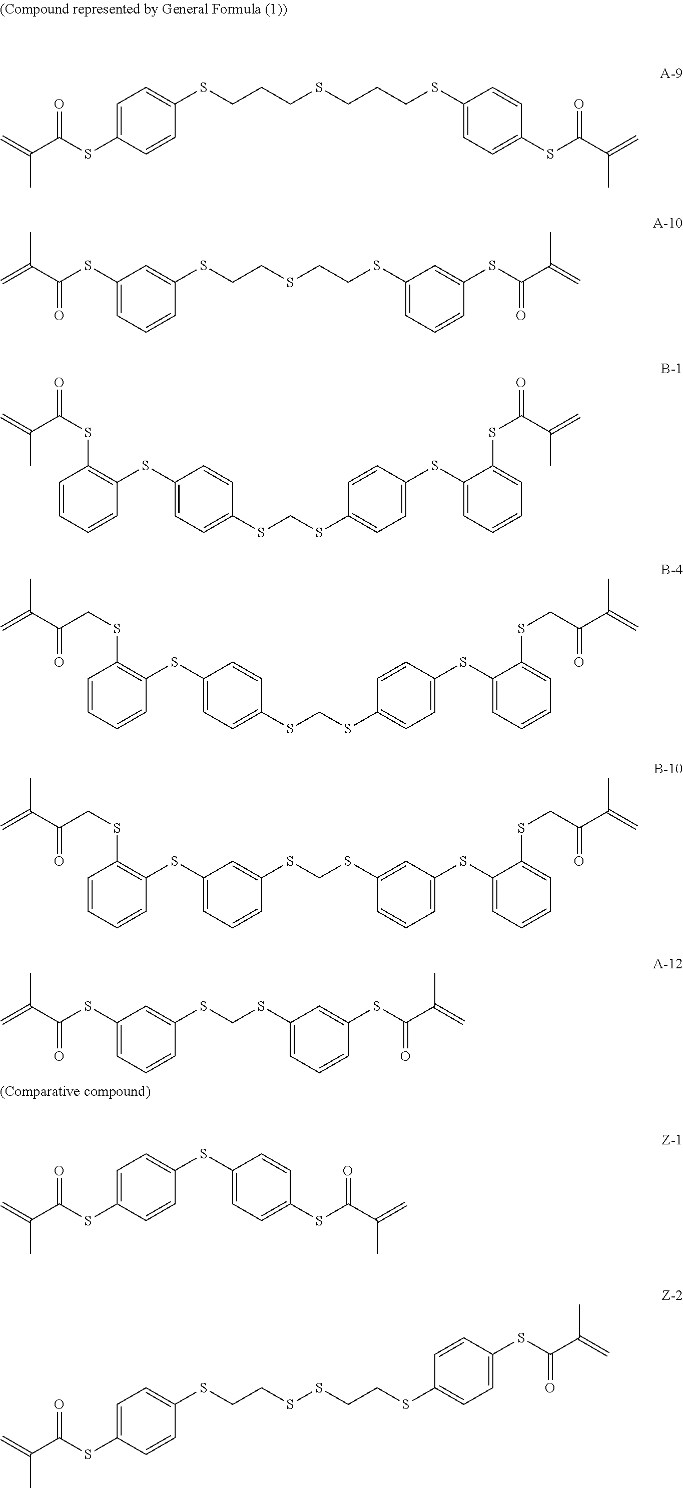

The comparative compound Z-1 was 4,4′-bis(4-methacryloylthiophenyl)sulfide disclosed in JP1990-193962A (JP-H2-193962A).

The comparative compound Z-2 was a compound No. 19 disclosed in Table 1 of JP1993-51412A (JP-H5-51412A).

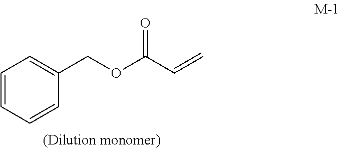

### (Photopolymerization Initiator)

IrgTPO: Irgacure TPO (product name, manufactured by BASF Japan, available as Omnirad TPO H (product name, manufactured by IGM Resins B.V.)); diphenyl(2,4,6-trimethylbenzoyl)phosphine oxide

From the results shown in Table 1, the following was found.

The comparative compound Z-1 was not the compound according to the embodiment of the present invention in that two phenylene groups in General Formula (1) of the present invention were linked through a group including a —S— bond, instead of the group represented by —S-L¹-S—. The curable resin composition containing the comparative compound Z-1 could not satisfy both high refractive index nD of the obtained cured product and suppression of the crystal precipitation of the composition. That is, in order to increase the refractive index nD of the obtained cured product to a desired level, in the curable resin composition No. c11 in which the content proportion of the comparative compound Z-1 was set to 80% by mass, crystals were precipitated from the composition in less than 24 hours of storage at 25° C., and the crystal precipitation of the composition was not sufficiently suppressed. In the curable resin composition No. c12 in which the content proportion of the comparative compound Z-1 was set to 50% by mass so that the crystal precipitation of the composition was sufficiently suppressed to be evaluated as “A”, the refractive index nD of the obtained cured product was less than 1.650, which did not satisfy the desired level.

The comparative compound Z-2 was not the compound according to the embodiment of the present invention in that two phenylene groups in General Formula (1) of the present invention were linked through a group including a —S—S— bond, instead of the group represented by —S-L¹-S—. In the curable resin composition No. c13 containing the comparative compound Z-2, the obtained cured product had deteriorated heat resistance.

With respect to these, in the curable resin compositions Nos. 101 to 108 containing the compound represented by General Formula (1) according to the embodiment of the present invention, the crystal precipitation of the composition was sufficiently suppressed, and the obtained cured product had a refractive index nD of 1.670 or more, resulting in excellent heat resistance. Among these, in the curable resin compositions Nos. 102 to 108 containing the compound according to the embodiment of the present invention, in which L¹ in General Formula (1) was an alkylene group having 1 to 5 carbon atoms or a group that, among one —CH₂— or two or more —CH₂— not adjacent to each other in an alkylene group having 1 to 5 carbon atoms, —CH₂— not adjacent to an S atom was substituted with —S—, the refractive index nD of the cured product could be increased while achieving both the suppression of the crystal precipitation of the composition and the heat resistance of the cured product, which was excellent. Furthermore, in the curable resin compositions Nos. 103 to 105, and 108 containing the compound according to the embodiment of the present invention, in which n1 and n2 in General Formula (1) were 1, the refractive index nD of the cured product could be further increased while achieving both the suppression of the crystal precipitation of the composition and the heat resistance of the cured product, which was more excellent.

In addition, as is clear from the comparison between the curable resin compositions Nos. 102 and 107 and the comparison between the curable resin compositions Nos. 105 and 108, in a case where, with regard to the compound represented by General Formula (1) according to the embodiment of the present invention, the content proportion of the compound was designed to be high concentration of 99.7% by mass in order to increase the refractive index nD of the cured product to be 1.690 or more, the excellent heat resistance of the cured product was maintained, and the crystal precipitation of the composition was also suppressed, which was excellent.

The present invention has been described with the embodiments thereof, any details of the description of the present invention are not limited unless described otherwise, and it is obvious that the present invention is widely construed without departing from the gist and scope of the present invention described in the accompanying claims.

What is claimed is:

1. A compound represented by General Formula (1), 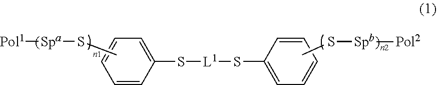
in the formula, L¹ represents an alkylene group or a group that, among one —CH₂— or two or more —CH₂—'s not adjacent to each other in an alkylene group, —CH₂— not adjacent to an S atom is substituted with —S—,
Sp^{a} and Sp^{b} represent a divalent aromatic hydrocarbon group or a divalent group formed by a combination of a divalent aromatic hydrocarbon group and an alkylene group,
Pol¹ and Pol² represent a (meth)acryloylthio group or a vinylthio group, and
n1 and n2 are 0 or 1.

2. The compound according to claim 1,
wherein L¹ has a number of linking atoms of 1 to 5, the linking atoms constituting a shortest molecular chain linking two sulfur atoms to which both terminals of L¹ are bonded.

3. The compound according to claim 1,
wherein n1 and n2 are 1.

4. A curable resin composition comprising:
the compound according to claim 1.

5. The curable resin composition according to claim 4,
wherein a content of the compound in the curable resin composition is 60% by mass or more.

6. The curable resin composition according to claim 4,
wherein the curable resin composition is for nanoimprint.

7. A cured product of the curable resin composition according to claim 4.

8. A diffractive optical element which is formed of the cured product according to claim 7, comprising:
a surface having a diffraction grating shape.

9. Augmented reality glasses comprising:
the diffractive optical element according to claim 8.
